(19) United States
(12) Patent Application Publication
(10) Pub. No.: US 2010/0051039 A1
(43) Pub. Date: Mar. 4, 2010

(54) Skin treatment device and method for exfoliation and cellulite reduction

(76) Inventor: Joella Ann Ferrara, Walnut, CA (US)

Correspondence Address: CHRISTOPHER D. HARRINGTON, SUITE 350, 5300 CORPORATE GROVE DRIVE, SE, GRAND RAPIDS, MI, 49512, US

(21) Appl. No.: 12/231,263

(22) Filed: Sep. 2, 2008

(51) Int. Cl. A61B 17/50; A61B 19/00

(52) U.S. Cl. 128/898; 606/131

(57) ABSTRACT

A novel device and method for use in providing multiple cosmetic treatments is disclosed, where a sanding tool is provided with sanding surfaces for use in imparting exfoliation, depilation, and cellulite treatments on the skin of a person. Select sanding surfaces are use for each of the exfoliation, depilation and cellulite treatments with the sanding action being generated by the orbital sanding effects of a palm sander. Specifically, a 100 grit (CAMI) sanding paper is used for exfoliation, a 180 grit (AMI) sanding paper is used for depilation, and the sander pad of the sanding tool of the present invention is used directly on the skin for cellulite reduction.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The present invention relates to a hand held device for the exfoliation of dead skin cells and for depilation of hair on the skin of a person and for the reduction of cellulite in a person. The present invention also relates to a method for exfoliation and depilation using the aforementioned device, and also to a method for the reduction of cellulite.

Custom and culture have long established various levels of preference or distaste for certain cosmetic features for humans. The range of such preferences and distastes has varied over the long history of mankind and there are probably established culturally based practices that are attributed to pre-historic times. In each stage of civilization's history though, it is well known that cultures have often evolved standards for certain cosmetic preferences and traits. One only has to visually compare and contrast the customs that defined the appearances of the ancient Egyptians versus the customs that defined the appearances of the members of high society and nobility during the period of time preceding the French Revolution. The point is that the cosmetic standards of people are sometimes dictated by the influence of cultural preferences. In some cases, though, there are hygienic reasons for a cosmetic preference, the simplest of which is the use of soap for cleansing one's face and body.

These standards, no matter how they are developed over time, do affect people and their behavior. In today's society one need not look any further than television broadcasts to see all of the commercials, infomercials, regular television shows highlighting certain “beautiful” people, and various other shows that offer assistance and advice on the pursuit of the ideal cosmetic standard. The level of importance that is placed on this aspect of our society is noteworthy because it underscores the novelty of any new approach that demonstrably affects a person's appearance in a manner that is in accordance with the prevailing cosmetic standards. The potentials for bringing new treatments or products to the cosmetic marketplace are so rewarding and so well scrutinized that the presentation of specific approaches that have not been tried before truly does represent a new and non-obvious offering. With this in mind, it is asserted that the present invention does indeed present a new and fresh approach to the cosmetic marketplace that addresses longstanding problems associated with the desire of people to reach certain cosmetic standards.

It has long been deemed in our modern U.S. culture that body hair is an indicia of cosmetic standards. At times facial hair on men has been in favor and then riot, and sometimes segments of society have favored chest hair for men suggesting it is a mark of virility. However, if one thing is true about the standards of any culture, it is that they are always subject to change. Thus facial and chest hair for men may not be in favor for a period of time, only to see the standards reversed after the passage of time, in one area, however, our culture has not wavered for some time and that is with respect to hair on women. This standard by most accounts applies to leg hair, underarm hair, and other body hair where it may appear.

In addition to hair removal, advocates and practitioners of cosmetic treatments will many times also seek to remove dead skin cells. This process, called exfoliation, is intended to reveal younger and healthier skin on the face and body of a person. As a mechanical process, exfoliation can be achieved through the use of micro-bead face scrubs that will remove the top layer of skin. A more extreme level of exfoliation is through dermabrasion which entails the use of a laser to remove upper layers of skin, although prior to the advent of laser technology, micro sanders were used (under anesthesia) to aggressively abrade the skin surface. Chemicals can be used to gain the same effect, the most common practices usually employ Salicylic acid (BHA) or Gylcolic acid (AHA).

There have been many efforts made in the prior art to attack the problem of unwanted hair, for both men and women. Numerous products ranging from chemical depilatories (sometimes called “hair creams”) to devices that casually resembles instruments of torture, to fairly common tools and practices such as would be the case with razors or shavers and the like. The present invention, however, relies on a hand tool to achieve depilation and aside from the use of tools that shave or cut, there are few hand tools that have been suggested for use on the body for the problems described herein. For instance, in U.S. Pat. No. 6,058,545 (Roach) a scrubbing tool is disclosed for use in scrubbing and massaging the back and lower body of a person. Roach does not teach a tool for the removal of hair, nor does the scrubber of Roach teach the utility of a device that can be used for exfoliation and for the reduction of cellulite.

In U.S. Pat. No. 4,149,530 (Gow) a vibrator is taught for use in massaging a person. The vibrator of Gow comprises a handle with one or more globes extending therefrom that impart a vibratory effect to the skin of a person. Gow does not teach any means for removing unwanted hair or any other specific and tangible effect. Similarly, the massager and washer of U.S. Pat. No. 5,385,532 (Shyu) is used to provide a massage effect while a person is in a tub or shower. Shyu does not teach anything regarding hair removal. The massager taught in U.S. Pat. No. 6,730,050 (Huang) advances the means by which massage action is imparted to paired massager heads, however, Huang does not produce any effect that would be useful for hair removal or the like. The hand-held buffer-moisturizer of U.S. Pat. No. 6,090,085 (Mehl, Sr, et al) is a slow rotating massager with provisions for supplying steam condensate directly to the Skin of a user. Mehl, Sr. does not teach anything regarding the removal of unwanted hair. The design for a massager, as shown in U.S. Design Pat. No. D476,087 does suggest a hand-held device although it is presumed that this device would merely be an extension of the types of massagers discussed within this paragraph.

In contrast to the foregoing devices of the prior art, the hair removal device taught in U.S. Pat. No. 4,935,024 (Dolev) does obviously cause hair removal. While this hand-held tool does have a rotating element, it functions by plucking the hairs from the skin of a user, e.g., capturing the hairs in between rotating discs and causing them to be pulled from their roots in the subcutaneous skin. This method is considered by some to be painful and can be irritating to certain individuals who may experience a reaction to the plucking action.

Lastly, in U.S. Pat. No. 4,592,170 (Hutchins, et al) an orbital sanding tool is disclosed which provides a means for attaching a piece of sanding paper to the bottom of the device and thereafter causing it to rotate. Hutchins is a tool that is conventionally directed towards the sanding of wood, metals or even plastics and is not thought of as a means for addressing hair removal or skin care in general. Aside from the fact that it is not suitable for use as a “hand tool” in the sense that a hand tool can be manipulated easily for a significant period of time with one hand, the sander taught by Hutchins drives a sanding head that is especially effective in ripping off layers of substrates such as wood or metal, as opposed to using the tool on the skin of a person.

The need for an efficient, effective and relatively painless tool for hair removal, exfoliation, and for the treatment of cellulite, persists even in the face of the many prior art devices. The usage of over-the-counter depilatories, while generally considered to be painless, is not as efficient or as pleasant as would be desired. The creams that are used necessarily evolve strong aromatic odors. In some cases the creams can provoke an allergic reaction. The usage of the creams is time consuming, not only in the application, but also because it requires time for the cream to work, and then it has to be removed and then the user has to wash the residues. The whole process is something that cannot be accomplished casually and requires some scheduling and forethought.

Ideally, a hair removal tool would be simple to use, easy to handle, and would not provoke a reaction or be considered painful in any way. These objectives are goals that have been met by the present invention, along with other benefits and attributes, and all of these are discussed in more detail herein.

### SUMMARY OF THE INVENTION

A novel hair remover and method for exfoliation, depilation and cellulite reduction utilizes a hand held, lightweight sanding tool comprising a sander body, a handle, a power supply, a sanding pad, select sand paper, and at least one spring clip for retaining the sand paper to the sanding pad. The sanding tool includes a motor and mechanism for imparting the sanding action to the sanding pad, which is transmitted to the sand paper when the sand paper is installed thereon.

The sand paper for use in the present invention is selected from different grit ranges for achieving different effects upon the skin of a person. For instance, sand papers at 100 grit (CAMI) are used for the exfoliation process and sand papers at 180 grit (CAMI) are used for depilation. Where the sanding tool includes a sanding pad that is comprised of a hard base, then the sanding tool can be applied to the skin of a person directly in order to cause the reduction of cellulite.

These and other attributes and features of the present invention will be discussed in more detail below.

### BRIEF DESCRIPTION OF THE DRAWINGS

FIG. 1 is an isometric view of a sanding tool of the type used in conjunction with the present invention, with sandpaper installed on the sanding tool.

FIG. 2 is a bottom view of the sanding tool depicted in FIG. 1, showing the sandpaper installed thereon in phantom.

FIG. 3 is a side elevational view of the sanding tool depicted in FIG. 1, and with the sandpaper installed.

FIG. 4 is an isometric view of two different sandpapers of the type that would be compatible for use in the sanding tool of the present invention, one corresponding to a grit of 100 (CAMI) and one corresponding to a grit of 180 (CAMI).

FIG. 5 is a partial cross section of the sanding tool depicted in FIG. 3, and as taken along Section Lines 5-5.

### DETAILED DESCRIPTION OF THE PREFERRED EMBODIMENTS

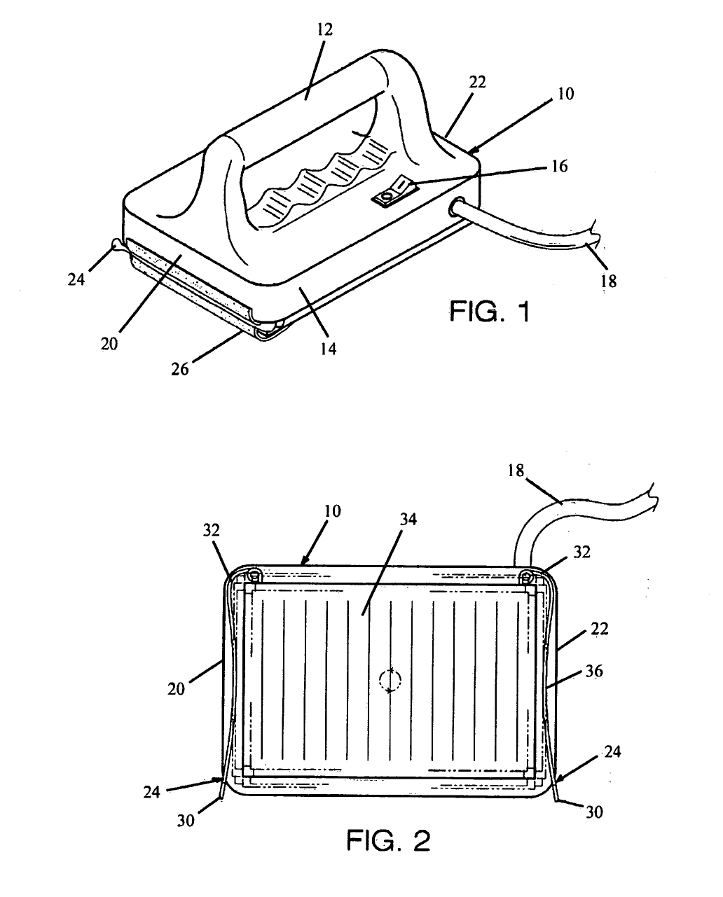

A sanding tool used in accordance with the present invention is shown in the drawings and is discussed in detail within this section. In FIG. 1, a sanding tool 10, known also as a palm sander, includes a handle 12, a sander body, 14, an on/off switch 16, a power cord 18, with sander front 20 and sander rear 22. Also shown is the spring clip 24 and with a piece of sanding paper 26 as installed onto the sanding tool 10.

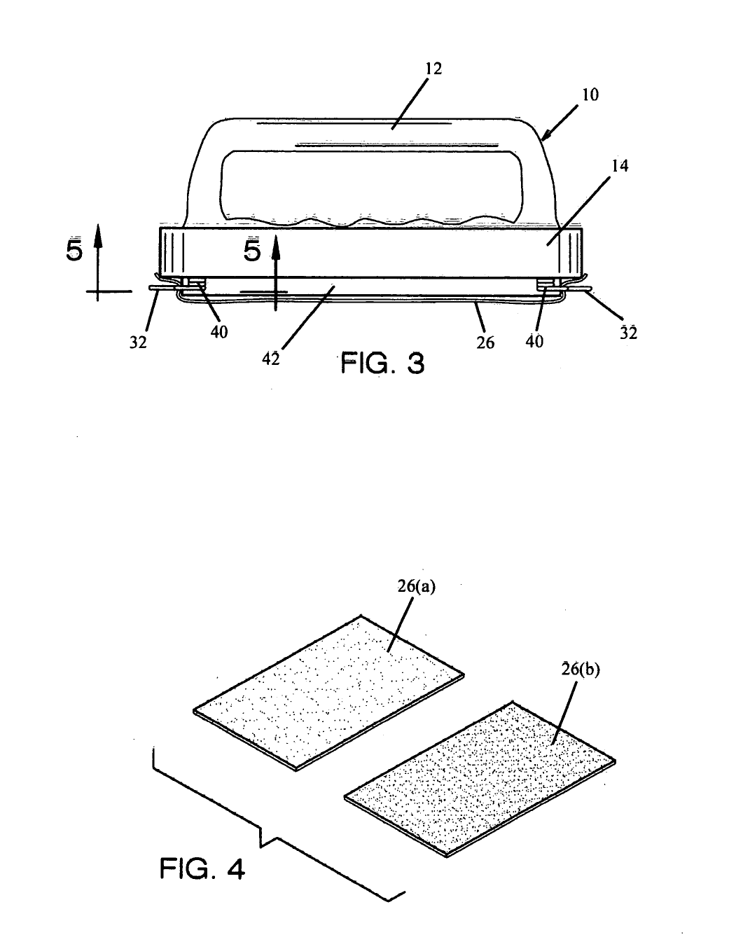

In FIG. 2, the sanding tool 10 is shown with the spring clip 24 which has a free end 30 and a fixed end 32 and a spring arm 36. The sanding tool 10 includes the sander pad 34. in FIG. 3, the fixed end 32 is shown with the coil springs 40 that are mounted into the sander base 42.

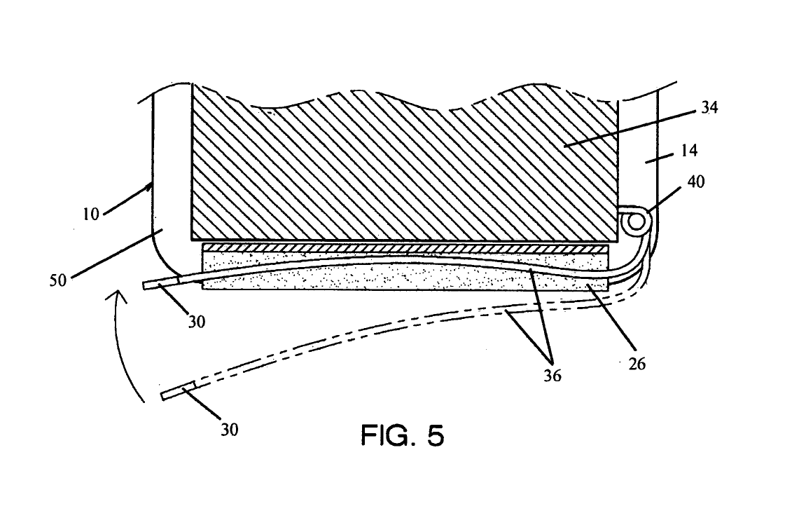

In FIG. 4, two different types of sanding papers are shown, where the sandpaper 26(a) is the 100 grit (CAMI) and the sandpaper 26(b) is the 180 grit (CAMI). The term “CAMI” is an acronym for the Coated Abrasives Materials Institute which is an industry group that provides guidance and that has implemented standards for sand paper among other materials. References to grit throughout this application will be with respect to the CAMI standards.

Lastly, in FIG. 5 a portion of the underside of the sanding tool 10 is shown with a piece of sandpaper 26 shown as installed and being held in place by the spring clip 24. Also viewable is the lower body edge 50 which runs along the periphery of the sander base 42 and where the two meet is formed a ninety degree angle.

The sanding tool is basically one type of a conventional tool that may be found in most hardware and home improvement stores. For the purposes of the present invention, however, the usage of a palm sander is necessary for the purposes of light weight and the ability to use it on the skin of a person. In addition, the palm sander is preferentially of the orbital type where it generates a circular sanding action. The sanding as shown in the drawings is powered by 110 V.A.C. line power as evidenced by the power cord. It is possible to use a cordless sanding tool, one that is typically powered by a rechargeable battery, however the duration of the potential use of a cordless is limited for long as the battery has sufficient power, beyond which it fails to work. Therefore the corded version is preferred in that it continues to work so long as there is line power.

As may be appreciated from the drawings, pieces of sanding paper can be installed onto the sanding tool by placing the sandpaper onto the sander pad and then pulling back on the free end of one spring clip, one end of the sandpaper is inserted into the space in between the spring clip arm and the sander base. The spring clip is under a tension that is exerted by the coil spring which is located near the fixed end of the spring clip. The sand paper is captured in place when the spring clip arm is released and the coil spring causes it to return back to its normal position. Viewing this situation in FIG. 5, the spring clip is shown in phantom in the open position where the sandpaper is insertable into the space in between the spring clip and the sander base, and the spring clip is also shown in regular format where it is at rest and in the closed position holding the sanding paper against the sander base. This same installation procedure is used for the other side of the sanding tool where the opposing spring clip is located.

There are some sanding tools that only have one spring clip, where one end of the sanding paper is merely inserted into a slot and is retained under tension when opposing end is clamped down using a spring clip. There are other ways to retain sanding papers to the sanding tools of the type involved in the present invention, and most of these may work, although the types that use sanding papers that use adhesive backings are to be avoided since some adhesive will still remain on the sander pad, which will interfere with the use of the sanding tool for the part of the procedures that will be discussed below for reducing cellulite.

The use of differing grit sizes generates different effects. The 100 grit sanding papers are considered to fall into the “fine” category. This is in the upper range of so-called “macrogrits” as opposed to the extremely fine micro-grits. Sanding papers in the 100 grit type category have been found to be very useful for exfoliation. Microsanders that have been used in the past for exfoliation have mainly been the province of plastic surgeons owing to the aggressive action they provide. An example of such a microsander is the Dremel tool which uses a sanding head that is insertable into a motorized body for driving it in a rotary manner. The usage of sanding papers in the range of 100 grit though have been found by experiment to be less aggressive and can be adequately handled by person in the cosmetology industry. The Dremel type of microsanders also have the drawback that they are relatively small which is of some benefit when they are used to abrade tattoos and callouses, but a device with a small sanding surface would become tedious for use as a tool for exfoliation on a larger scale where more surface area is involved. Usage of grits in the ISO (international Standards Organization) ranges of P100 to P120 would also do service in exfoliation although the standard 100 grit is preferred.

Grit size of 180 has been found to be surprisingly effective for hair removal when used in the orbital palm sander of the type disclosed herein. This grit is considered to be in the very upper end of the “very fine” category and like the sand paper in the previous discussion, it is still considered to be part of the macrogrit type of sandpaper. In this instance the very fine sanding action of the 180 grit paper will abrade and cause the hair shaft to be severed. Once this happens the hair will separated from the skin yet the sanding tool is still applied to further abrade the hair shaft until it is trimmed down to the skin surface. With respect to ISO graded sanding paper, grit of the same size essentially, P180 would be useable in the hair removal process of the present invention.

The last mode of use of the sanding tool of the type disclosed above, is for the reduction of cellulite. Typically this occurs in the thighs and the buttocks and is a persistent cosmetic problem for many women. There are no real cures for cellulite although some treatments have a demonstrable cosmetic effect that will reduce the cellulite appearance. In the present case, the sanding tool of the present invention, when used without sanding papers, will beneficially reduce the appearance of cellulite on a person. The effect is best when the sander pad is comprised of a hard rubber surface or similar plastic type surface. Palm sanders will typically come with such surfaces and they possess a small amount of frictional engagement that is used to assist in retaining the sanding paper to the sander pad. However, when this same surface is directly applied to skin that has the cellulite appearance, the orbital action of the sanding tool provides a deep tissue massage that impacts the cellulite structure.

It has been found through experimentation and research, that the sanding tool of the types described herein, in conjunction with the selection of the type of sanding surface to be applied, e.g., sanding paper in the range of 100, 180 or the use of the surface of the sander pad, produces a combination of effects that are useful to persons seeking to gain certain cosmetic results. Thus a method for the use of the sanding tool to further this development has been refined and is comprised of the following steps.

Depending on which process is to be used, a selection is made of the type of sanding surface that will be needed. The sanding surface, if it is a sanding paper, is installed on the sanding tool. The sanding tool is then plugged into a power source (unless the sanding tool is of the cordless type). The sanding surface is applied to the skin of a person in the area where the desired cosmetic treatment is to be imparted. With the sanding tool switched to the “on” position, the sanding surface contacts the person's skin and the sanding tool is guided by the user over the area to be treated. The user does not have to provide a circular motion to achieve effective treatment, although the person may find that the use of a circular motion is comfortable. The orbital effect imparted by the sanding tool is the main factor in achieving a result using the specified sanding surfaces. Once the desired cosmetic effect has been obtained the sanding tool is de-energized by turning the switch to the “off” position. Following this, the same procedure may undertaken again with the selection of a second sanding surface, and yet once more thereafter with the selection of a third sanding surface.

It may be that a person will receive all three forms of treatment, exfoliation, hair removal, and cellulite reduction. In such a case, the process has been simplified through the use of the present invention since all three forms of treatment can be obtained with a single piece of equipment.

The teachings of the present invention may be varied without departing from the spirit and scope of the present invention. Nothing within this specification is intended to act as or should be construed as a limitation on the invention as disclosed.

I claim:

1. A sanding tool for use in applying multiple cosmetic treatments to a person, specifically an electrically powered palm sander with orbital sanding action, and where the palm sander includes a sander pad, the palm sander compatible with the receipt of different sanding surfaces for achieving the different cosmetic treatments, the sanding tool comprising:
A sanding surface selected from the group of 100 grit sanding paper, 180 grit sanding paper, or the sander pad surface; and,
Where the use of 100 grit sanding paper is used for exfoliation, and where the use of 180 grit is used for depilation, and where the use of the sander pad surface is used for cellulite reduction.

2. The sanding tool of claim Number 1, where the sanding tool is powered 110 V.A.C.

3. The sanding tool of claim Number 1, where the sanding tool is powered by a rechargeable battery.

4. The sanding tool of claim Number 1, where the sanding papers are retained to the sanding tool by at least one spring clip.

5. A method for imparting multiple cosmetic treatments to the skin of a person, the steps of the method comprising:
Acquiring a palm sander with orbital action, with a sander pad capable of receiving different sanding papers;
Selecting a sanding surface for use in the cosmetic treatment to be imparted to the skin of the person;
Configuring the palm sander for the selected sanding surface;
Energizing the palm sander;
Applying the selected sanding surface to the skin of the person until the desired cosmetic condition has been obtained; and,
Removing the sanding surface from the skin of the person and de-energizing the palm sander.

6. The method for imparting multiple cosmetic treatments in accordance with claim Number 5, where the selection of sanding surfaces is from the group of 100 grit sanding paper, 180 grit sanding paper, or the sander pad surface, where the use of 100 grit sanding paper is used for exfoliation, and where the use of 180 grit is used for depilation, and where the use of the sander pad surface is used for cellulite reduction.

7. The method for imparting multiple cosmetic treatments in accordance with claim Number 5, including the additional steps:
Following the removal of the sanding surface from the skin of the person and the de-energization of the palm sander, reconfiguring the palm sander for a second selected sanding surface;
Energizing the palm sander;
Applying the selected sanding surface to the skin of the person until the desired cosmetic condition has been obtained; and,
Removing the sanding surface from the skin of the person and de-energizing the palm sander.

8. The method for imparting multiple cosmetic treatments in accordance with claim Number 7, including the additional steps:
Following the removal of the sanding surface from the skin of the person and the de-energization of the palm sander, reconfiguring the palm sander for a third selected sanding surface;
Energizing the palm sander;
Applying the selected sanding surface to the skin of the person until the desired cosmetic condition has been obtained; and,
Removing the sanding surface from the skin of the person and de-energizing the palm sander.

9. A method for imparting multiple cosmetic treatments to the skin of a person, the steps of the method comprising:
Acquiring a palm sander with orbital action, with a sander pad capable of receiving different sanding papers;
Selecting a sanding surface for use in the cosmetic treatment to be imparted to the skin of the person where the selection of sanding surfaces is from the group of 100 grit sanding paper, or 180 grit sanding paper, where the use of 100 grit sanding paper is used for exfoliation, and where the use of 180 grit is used for depilation;
Configuring the palm sander for the selected sanding surface;
Energizing the palm sander;
Applying the selected sanding surface to the skin of the person until the desired cosmetic condition has been obtained; and,
Removing the sanding surface from the skin of the person and de-energizing the palm sander.

10. The method for imparting multiple cosmetic treatments in accordance with claim Number 9, including the additional steps:
Following the removal of the sanding surface from the skin of the person and the de-energization of the palm sander, reconfiguring the palm sander for a second selected sanding surface;
Energizing the palm sander;
Applying the selected sanding surface to the skin of the person until the desired cosmetic condition has been obtained; and,
Removing the sanding surface from the skin of the person and de-energizing the palm sander.

11. The method for imparting multiple cosmetic treatments in accordance with claim Number 9, where the group of sanding surfaces further includes the use of the sander pad surface is used for cellulite reduction.

12. The method for imparting multiple cosmetic treatments in accordance with claim Number 11, including the additional steps:
Following the removal of the sanding surface from the skin of the person and the de-energization of the palm sander, reconfiguring the palm sander for a second selected sanding surface;
Energizing the palm sander;
Applying the selected sanding surface to the skin of the person until the desired cosmetic condition has been obtained; and,
Removing the sanding surface from the skin of the person and de-energizing the palm sander.

13. The method for imparting multiple cosmetic treatments in accordance with claim Number 12, including the additional steps:
Following the removal of the sanding surface from the skin of the person and the de-energization of the palm sander, reconfiguring the palm sander for a third selected sanding surface;
Energizing the palm sander;
Applying the selected sanding surface to the skin of the person until the desired cosmetic condition has been obtained; and,
Removing the sanding surface from the skin of the person and de-energizing the palm sander.
